(19) United States
(12) Patent Application Publication
(10) Pub. No.: US 2026/0306065 A1
(43) Pub. Date: Oct. 8, 2026

(54) OPERATOR-INDEPENDENT ULTRASOUND IMAGING

(71) Applicant: THE JOHNS HOPKINS UNIVERSITY, Baltimore, MD (US)

(72) Inventors: Aliaksei PUSTAVOITAU, Baltimore, MD (US); Amir MANBACHI, Baltimore, MD (US); Kaushik SAMPATH, Riverdale, MD (US); James WISSMAN, Laurel, MD (US); Haley Gilbert ABRAMSON, Baltimore, MD (US); Joshua PUNNOOSE, Baltimore, MD (US); Kelley KEMPSKI LEADINGHAM, Baltimore, MD (US)

(73) Assignee: THE JOHNS HOPKINS UNIVERSITY, Baltimore, MD (US)

(21) Appl. No.: 19/482,406

(22) PCT Filed: May 7, 2024
(86) PCT No.: PCT/US2024/028125

§ 371 Date: Nov. 7, 2025

Related U.S. Application Data
(60) Provisional application No. 63/501,752, filed on May 12, 2023.

(51) Int. Cl. A61B 8/00

(52) U.S. Cl. CPC A61B 8/5223; A61B 8/4227; A61B 8/4236; A61B 8/4483; A61B 8/488; A61B 8/585

(57) ABSTRACT

The system includes a wearable device that is operator-independent, and can be utilized in a multiple-transducers configuration to monitor multiple sites simultaneously. The method for monitoring a medical condition of a patient includes receiving an indication that a transducer array is placed at a position in proximity to a body part of the patient to be scanned, the transducer array including a plurality of transducer array elements, the transducer array being a part of a wearable device. The method also includes producing transducer data by scanning the body part. The method also includes conducting a type of test using the transducer data, wherein the transducer array is configured to operate without supervision.

### CROSS-REFERENCE TO RELATED APPLICATIONS

This application is the national stage entry of International Patent Application No. PCT/US2024/028125, filed on 7 May 2024, and published as WO 2024/238202 A1 on 21 Nov. 2024, which claims the benefit of U.S. Provisional Application No. 63/501,752, filed 12 May 2023, which are hereby incorporated by reference herein in their entireties.

### FIELD OF THE DISCLOSURE

Materials, components, and methods consistent with the present disclosure are directed to the use of ultrasound to monitor anatomical structures.

### BACKGROUND OF THE DISCLOSURE

Ultrasound imaging as a medical diagnostic tool can image organs as well as fluid flow in the human body. An ultrasound system, including an array of elements that emit and receive ultrasonic waves, can generate multi-dimensional images and grayscale images or provide measurements of velocities of blood and fluid (Doppler mode). The velocities are derived from changes in phase of the received ultrasonic waves resulting from the transmission of streams of high-frequency sound waves and analyzing signals reflected from circulating red blood cells. The position of an ultrasound transducer affects the strength of the Doppler signal due to blood flow, so that manual positioning results in variable signal strength.

The ultrasound transducer emits sound waves in discrete pulses into tissue, and between pulses, listens for returning pulses. When the tissue is encountered, a portion of the sound waves is reflected back to the transducer, the portion being dependent upon characteristics of the tissue. For example, the depth of the tissue is related to the time between pulse emission and pulse return. An image (referred to as a B-mode or grayscale image) can be created based on the number of returning pulses and their return time. The time between pulse emission and pulse return decreases with higher ultrasound probe frequencies.

Point-of-care ultrasound (POCUS) is transforming the practice of medicine by allowing immediate decision making by the clinician at the patient's bedside. While recent development of high-quality, portable devices in the price range of $5-10K (many of which connect to smartphones and tablets) has led to POCUS gaining widespread use, ultrasound imaging still relies on manually-operated probes to obtain diagnostic quality images. Therefore, despite their operation in real-time, these images fail to capture developing pathological processes. An operator-independent ultrasound transducer could perform continuous, long-term monitoring of acutely ill or injured patients. For example, early prediction and detection of deep vein thrombosis (DVT) in the lower extremities, a precursor of often-deadly pulmonary embolism (PE), could be accomplished. The CDC reports approximately 900,000 annual cases in the United States of DVT/PE, of which 100,000 are fatal, and estimates incurred yearly healthcare costs at $10 billion.

Prolonged periods of immobility, such as extended stays in the intensive care unit (ICU), increase the risk of clot formation in the deep veins of the lower limb. Up to 10% of surgical ICU patients are diagnosed with DVT when they are admitted to the ICU, and the other surgical ICU patients have up to 40% risk of developing DVT while in the ICU due to hypercoagulability and stasis. Many ICU patients receive a blood thinner regimen to prevent clot formation, though anticoagulants may lead to spontaneous bleeding at the surgical site. This bleeding can be very dangerous in, for example, the brain or spinal cord; therefore, neurosurgery patients are often not given thromboprophylaxis. Undetected, DVT can result in detrimental sequelae, including PE, respiratory failure, shock, and death. Improved monitoring for DVT in ICU patients may help ensure early DVT detection and treatment. There is an unmet need for a wearable sensor for continuous, long-term monitoring of acutely ill or injured patients in the ICU to predict, detect, and monitor the development of and response to DVT in the lower extremities. The CDC's report also emphasizes the need for improved monitoring of patients at risk for developing DVT. DVT is typically evaluated using: 1) B-mode ultrasound imaging to ensure that the deep veins are not clotted (they compress with light pressure), and 2) spectral and color Doppler imaging to view healthy blood flow (see FIG. 1). Whole-leg evaluation of both lower extremities can take up to forty minutes, but it has been shown that two-point compression tests, which focus solely on the femoral and popliteal regions (groin and knee, respectively), produce similar results in five minutes. Whole-leg duplex and 2D compression ultrasound tests are performed intermittently, due to the need for costly equipment and the presence of a sonographer or a physician. In some clinical settings, ICU patients are monitored for DVT weekly. The extended time gaps between DVT examinations increases its risk. It also limits the data collected and, therefore, an understanding of the physiological changes in blood flow that lead to clot development. A hands-free, wearable ultrasound probe placed at the femoral and/or popliteal veins on each leg (2-4 ultrasound patches) could facilitate rapid, continuous observation of veins and venous blood flow without manual intervention, reducing the need for on-site presence of a healthcare professional.

Abramson et al., Towards a Universal Device for Point-of-Care Medicine: A Custom Transducer for Long-term Monitoring of Local Vascular Flow via Ultrasound Imaging, Proceedings of the 2022 Design of Medical Devices Conference, Apr. 11-14, 2022, Minneapolis, MN (Abramson), describe a custom transducer for long-term monitoring of local vascular flow via ultrasound imaging. The commercially-available transducer of Abramson's disclosure, providing 2-D visualization via brightness mode (B-mode) ultrasound and flow detection via pulse-wave (spectral) Doppler imaging, is coupled with a semi-conformal outer casing to lay on any surface and operate without further handling after placement. This device can detect sudden changes in local vascular output, and capture human-level blood velocities.

U.S. Pat. No. 10,792,011 entitled Systems and Methods for Hand-Free Continuous Ultrasonic Monitoring, Toume et al., issued Oct. 6, 2020, describes a hands-free ultrasonic assembly that mounts at the suprasternal notch and includes receive and transmit beam formers and associated signal processing. Both mechanical adjustments to the position of the transducer, located in a cradle that is positioned at the suprasternal notch of the patient, and electronic adjustments to the beam are possible in this disclosure. An acoustic coupling material is situated between the device and the patient's skin. This can be applied by a wearable device that is also connected to other sensors whose data values can be used to guide the timing of the ultrasonic signals, or for other reasons. The transducer can operate concurrently or alternately in Doppler and B-mode. Automatic detection of the target section is accomplished by knowing the expected size/shape of the target section. Motion mode, color Doppler, and spectral Doppler can also be used. B-mode can be used to locate the target section, and Doppler mode can be used to measure the velocity at the target.

U.S. Pat. No. 11,020,092 entitled Multi-site Concurrent Ultrasound Blood Flow Velocity Measurement for Continuous Hemodynamic Management, Xu et al., issued Jun. 1, 2021, describes a Doppler ultrasound device having multiple transducer patches that can determine multiple blood flow velocities concurrently. The patches can be attached to the skin by adhesives or by a wrap. The wearable device includes an array of transducers whose average or maximum blood flow measurement determines the blood flow velocity. Thus, less precise alignment is used to achieve adequate measurement results. The multiple transducers are connected by wire to a control system.

What is needed is an operator-independent, low-profile ultrasound device for continuous scanning of, for example, but not limited to, venous vasculature and blood flow, on the timespan of hours to weeks. In an exemplary application, early detection of DVT that would help prevent PE in susceptible patients could be accomplished. What is needed is a device that provides clinicians and researchers with the ability to observe anatomic structures and continuous flows, without exceeding thermal or mechanical thresholds in accordance with safety limitations of regulatory aspects (e.g. accumulation of excessive heat). What is needed is an ultrasound device capable of grayscale and spectral Doppler ultrasound imaging, that can adhere to the evaluation site, maintain accuracy under repeated compression, and avoid overheating during use. What is needed is a device that can automatically alert clinicians of sudden changes in patient heath status. What is needed is automated beam steering of ultrasound waves. What is needed is an operator-independent, wireless wearable device for imaging any body part and monitoring health characteristics.

### SUMMARY

In one aspect, embodiments in accordance with the present disclosure include a wearable device that is operator-independent, and can be utilized in a multiple-patch configuration to monitor multiple sites simultaneously.

Possible applications of such a device include DVT early detection and monitoring. Historical information of in vivo studies of DVT enable identification of clot formation which is associated with early signs and progression of DVT. Existing studies have been limited by the lack of a continuous monitoring device at a site interest before disease onset. The device, in accordance with embodiments of the present disclosure, when placed at or near the site of interest, before the conditions leading to DVT develop, monitors the site of interest throughout the development of a blood clot, if that occurs. Some or all of the following metrics can be used to determine the status and progression of a clot: (i) appearance of the blood, (ii) spontaneous echo contrast, (iii) respirophasic variation in the blood flow, (iv) Doppler flow, (v) spectral Doppler imaging of peak velocity and average velocity, (vi) blood flow and size augmentation of veins with sequential compression device activation, and (vii) compressibility of the vein.

The current standard of care for DVT diagnosis involves a compression test with an ultrasound probe to assess whether the femoral and popliteal veins collapse (no clot present) or not (clot present), and the use of spectral and color Doppler ultrasound imaging to view venous blood flow. Placement of a wearable ultrasound patch, in accordance with embodiments of the present disclosure, directly over the femoral and popliteal veins facilitates continuous ultrasound monitoring of venous blood flow using spectral Doppler. Typically, healthy flow through a vein exhibits laminar flow whereby the red blood cells follow smooth, approximately parallel paths through the vessel walls. Unhealthy changes in the blood flow can be visualized as chaotic, turbulent flow creating a smoky appearance on grayscale imaging. Laminar and turbulent flow may show distinguishing features in spectral Doppler imaging. Specifically, turbulent flow may be associated with spectral broadening. The wearable device in accordance with embodiments of the present disclosure can eliminate the need for a probe operator to manually perform blood flow and compression tests by enabling execution of signal processing instructions that analyze data captured through non-compressional Doppler imaging of the femoral and popliteal veins. To augment the non-operator-dependent DVT diagnostics, a device in accordance with embodiments of the present disclosure is configured to perform compression testing. In an aspect, the compression testing takes the form of a smart tourniquet with ultrasound sensing capabilities. The smart tourniquet applies, without manual intervention, positive pressure for compression testing. The measure of positive pressure can provide information with respect to patient health status. For example, compression testing can be used to predict blood clot formation, monitor blood flow, and act as a heating pad, among other uses.

Existing ultrasound devices that perform continuous monitoring are limited due to overheating. Overheating can lead to inaccurate readings and potential burns or irritation to a patient's skin. The device of the present disclosure is configured to avoid heat deposition by using both computational approaches such as, for example, but not limited to, lowering duty cycles, pause periods, frame rates, and signal processing, and hardware approaches for example, but not limited to, employing a heat-conductive casing for the electronics.

The device in accordance with embodiments of the present disclosure is a conformal wearable device that provides real-time anatomical imaging (e.g., B-mode images) and real-time functional imaging (e.g., flow sensing using spectral Doppler). Existing conformal ultrasound sensors that provide imaging or blood pressure sensing do not provide both B-mode imaging and spectral Doppler simultaneously. The device of the present disclosure enables the prediction and detection of DVT earlier, potentially saving up to 100,000 lives each year in the United States alone. The device of the present disclosure is configured to monitor vascular flow to detect disruptions. The device is used in applications requiring portable, accessible, inexpensive, and non-radiating monitoring.

The device or patch according to embodiments of the present disclosure includes a grid of ultrasound transducers associated with a flexible, adherent, conductive material that conforms to the patient's body. When the device is placed in the general area of a region of interest, the device executes a calibration step in which individual ultrasound transducers are fired to locate vessels of interest. From the images obtained from the ultrasound transducers, the transducer providing a view of the vessels that meet pre-selected criteria is identified. For example, for images of possible DVT, in the thigh, an axial view of two vessels look like circles that represent the femoral vein and femoral artery. These have maximum flow velocity at the exact right angle. In other words, the one view would be of blood vessels in short axis (circles), arterial and venous, and the number and relative position of those vessels depend on anatomic location of the ultrasound transducer. In an aspect, a machine learning model that is trained to determine which ultrasound unit meets the pre-selected criteria is fed the images from the ultrasound transducers. The selected transducer is used for subsequent imaging, thus reducing the power requirements of the device and further reducing risk of overheating.

In one aspect, embodiments in accordance with with the present disclosure include a conformal, stretchable, washable, flexible, and/or rigid substrate of bioelectronics, a wearable ultrasound patch, capable of continuous monitoring. In an aspect, the wearable device is operator-independent and can be utilized in a multiple-patch configuration to monitor multiple sites simultaneously. In an aspect, the wearable device, in conjunction with supporting components of the system of the present disclosure, can be used to provide grayscale imaging in which an anatomical structure and its function are central biomarkers. In an aspect, in a multiple wearable device configuration, the system can be utilized to integrate data and reconstruct multi-dimensional anatomy in real time.

With respect to operator-independent operation, in an aspect, for example, no operator is at a patient's bedside after the device is attached to the patient's skin. In an aspect, once attached to the skin, the device remains substantially in place for a pre-selected amount of time during which the device transmits an ultrasound beam at prespecified intervals and optimal angle to sample the size of the anatomic structures, the function of the structure over time, and blood/tissue movement as used by a specific clinical application.

With respect to simultaneous scanning in multiple locations, in an aspect, since an operator does not manipulate the device, multiple devices can be utilized simultaneously for monitoring. For example, multiple devices can be used to monitor venous structures and blood flow for early detection of DVT. In an aspect, four devices are used, two on each of the femoral area and the popliteal area.

With respect to wearability, in an aspect, the device remains substantially in place due to adhesion to the skin of the patient or other means for holding the device in place, such as a physical restraint on the device, enabling image production even as the body moves.

In an aspect, configurations in accordance with the present disclosure include color or spectral Doppler capabilities, enabling physiological monitoring. In these configurations, flow sensing is a central biomarker, among others. In an aspect, the components of the present disclosure can enable, either individually or in conjunction with each other, photoacoustic imaging, thermometry, and/or pressure/force sensing. In an aspect, the system can include wired, wireless, and/or battery-powered components. In an aspect, the system can include components that are manufactured from ceramics (e.g., lead zirconate titanate (PZT)), polymers (e.g., polyvinylidene fluoride (PVDF)), a composite (including both ceramics and polymers), and/or capacitive transducers (e.g., capacitive micromachined ultrasonic transducer (CMUT)). In an aspect, the system of the present disclosure can be incorporated into materials that are safe, durable, and easy to clean such as, for example, but not limited to, textiles, polymers, liquid silicone rubber (LSR), ethylene propylene diene monomer (EPDM), and polytetrafluoroethylene (PTFE) that can enable, for example, but not limited to, smart clothing and smart tourniquets.

In an aspect, embodiments in accordance with the present disclosure include a mechanism for optimizing ultrasound beam steering, data acquisition, and display. Beam steering as described herein refers to an alteration of the angle of an ultrasound beam with respect to a transducer without moving the transducer, enabling a specific target to be inspected from multiple angles by a single transducer without moving the transducer. In an aspect, beam steering is enabled automatically, without manual manipulation of the ultrasound device.

In an aspect, embodiments in accordance with the present disclosure include artificial intelligence (AI) techniques for ensuring imaging and measurements, identification of measurements and patterns consistent with a particular pathology of the anatomy, and multi-dimensional view reconstruction of anatomic structures. In an aspect, embodiments in accordance with the present disclosure ensure consistent imaging and measurements across similar anatomic structures by monitoring and comparing physiologic information over time. In an aspect, images are initially collected in an optimal plane, and subsequently images are collected within the same optimal plane and velocities are measured at the same angle of incidence of the ultrasound beam on the anatomic structure. In an aspect, AI can be used to determine the optimal plane and to identify measurements and patterns consistent with either normal or abnormal anatomy, or function of the structure. In an aspect, the system can include an alarm system consistent with high risk pathology or patient condition change. In an aspect, the system can enable multi-dimensional reconstruction of anatomic structures from the collection of multiple views of the same structure.

In an aspect, embodiments in accordance with the present disclosure include systems that can be used for both therapeutic and/or diagnostic ultrasound purposes. In an aspect, variations in power/voltage, frequency, duty cycle and sanitation time can enable various use cases and applications of the system of the present disclosure.

In an aspect, embodiments in accordance with the present disclosure include components that can be utilized in coordination with systems and methods for triaging and/or prioritizing patients during mass casualties. For example, the system can be used to establish the presence of shock in a patient, and establish the causes of shock including pneumothorax, pericardial tamponade, hypovolemia, hemothorax and hemoperitoneum. By operating the device at different frequencies or in different modes, changing the transducer array, and/or placing multiple devices in different locations on the patient's body, the image will look different and be interpreted differently. Further algorithms can be developed to interpret these images. For example, in lung ultrasound imaging, the sonographer looks for artifacts. In an aspect, embodiments in accordance with the present disclosure include systems that can be utilized in coordination with systems and methods (1) to assess the causes of cardiopulmonary arrest and the effectiveness of cardiopulmonary resuscitative measures by changing the frequency and size of the fabricated wearable patch and/or by changing the transducer array, and/or (2) to triage patients, and/or (3) to monitor for deep venous thrombosis (DVT) development and effectiveness of therapies of DVT prevention and treatment. Such monitoring can be applied to ambulatory and hospitalized surgical patients, ill patients, and long-distance travelers. In an aspect, embodiments in accordance with the present disclosure include components that can be utilized in coordination with systems and methods (1) to evaluate patency and flow through reconstructed or surgically repaired blood vessels, (2) to triage and monitor patients in remote locations and austere environments, (3) to assess and guide hemodynamic resuscitation and respiratory support during a perioperative period, (4) to monitor and optimize mechanical ventilation of surgical, ill, and injured patients, (5) to evaluate anatomy and support airway management of surgical, ill, and injured patients, (6) to triage and monitor surgical, ill, and trauma patients at risk for acute kidney insufficiency, (7) to assess the effectiveness of renal replacement therapy in patients with acute renal failure and end stage renal disease, (8) to promote injured tissue healing, (9) to evaluate physiologic and pathologic processes and prediction of the transition to pathologic states, (10) to detect, at an early stage, pathologic abnormalities to minimize impact on a patient's well-being, and (11) to assess the responses to interventions (pharmacologic and other) during managing of acutely ill or injured patients, among other exemplary uses by acquiring medical images. Multiple devices placed around the body can view the legs, lungs, heart, kidneys, etc. Fine-tuned algorithms ease the transition across anatomies. Continuous imaging assesses changes in observed parameters in response to interventions, thus evaluating the impact of interventions in real time

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method for monitoring a medical condition of a patient. The method includes receiving an indication that a transducer array is placed at a position in proximity to a body part of the patient to be scanned, the transducer array including a plurality of transducer array elements, the transducer array being a part of a wearable device. The method also includes producing transducer data by scanning the body part. The method also includes conducting a type of test using the transducer data, wherein the transducer array is configured to operate without supervision. Other embodiments of this aspect include corresponding computer systems, apparatus, and computer programs recorded on one or more computer storage devices, each configured to perform the actions of the methods.

Implementations may include one or more of the following features. The method as where producing transducer data by scanning the body part may include: automatically directing the transducer array to sweep an ultrasound beam across the body part when the indication is received; capturing images as the ultrasound beam is sweeping; locating an image of interest from the captured images, a position of the ultrasound beam where the image of interest was collected, and a collecting transducer array element from the plurality of transducer array elements that collected the image of interest, the image of interest being based on the type of test, the collecting transducer array element centering a tissue of interest; and moving the ultrasound beam to the position where the image of interest was collected, where the position is associated with a beam steering angle. The method as may include: saving the beam steering angle for future grayscale imaging of the patient. The method as may include: enabling collecting Doppler images in proximity to the position using a Doppler angle that is different from but related to the beam steering angle. Locating the image(s) of interest may include: providing the image to an inference engine; and receiving information from the inference engine, the information enabling identification of the image(s) of interest. The method as may include: providing the image of interest to a predictive model, and receiving a diagnosis and a probability that the diagnosis is correct from the predictive model. The method as may include: using the diagnosis to activate/deactivate selective of the plurality of transducer array elements. The proximity position may include a popliteal fossa. The wearable device may include a backing layer, automatic calibration, wireless communications electronics, one or more sensor, and/or a tourniquet. Implementations of the described techniques may include hardware, a method or process, or computer software on a computer-accessible medium.

One general aspect includes a wearable ultrasound device for continuous monitoring of tissue. The wearable ultrasound device includes a transducer array including a plurality of transducer array elements and a plurality of subdice elements; a thermal sensor configured to monitor a temperature of the wearable ultrasound device having a pre-selected thickness and spacing between elements. The device also includes communications electronics receiving control information to control the plurality of transducer array elements. The device also includes where the control information enables continuous monitoring of the plurality of transducer array elements. The device also includes where the communications electronics transmits data from the thermal sensor and the plurality of transducer array elements to a processor. Other embodiments of this aspect include corresponding computer systems, apparatus, and computer programs recorded on one or more computer storage devices, each configured to perform the actions of the methods.

Implementations may include one or more of the following features. The wearable ultrasound device as may include: an acoustic lens. The control information may include beam steering information including instructions to: automatically direct the transducer array to sweep an ultrasound beam across the tissue when the wearable ultrasound device is affixed to a surface, and save the beam steering angle for grayscale imaging of a patient. The beam steering information may include instructions to: capture images as the ultrasound beam is sweeping; locate an image of interest from the captured images, a position of the ultrasound beam where the image of interest was collected; identify transducer array elements from the plurality of transducer array elements that collected the image of interest, where the image of interest is based on a type of test, where the identified transducer array elements center an area of interest; and move the ultrasound beam to the position where the image of interest was collected, where the position is associated with a beam steering angle. The control information enables collecting Doppler images in proximity to the position using a Doppler angle that is different from but related to the beam steering angle. The communications electronics may include wireless electronics. The control information may include: duty cycle information for the transducer array elements, the duty cycle information based on data from the thermal sensor. Implementations of the described techniques may include hardware, a method or process, or computer software on a computer-accessible medium.

One general aspect includes a system for continuous ultrasonic monitoring of a wearable device configured to collect ultrasound data from a user wearing the wearable device, the wearable device including: a transducer array including a plurality of transducer array elements; a thermal sensor configured to monitor a temperature of the wearable device; and communications electronics receiving control information for the transducer array elements, the control information enabling the continuous ultrasonic monitoring, the communications electronics transmitting data from the thermal sensor and the transducer array elements to a processor, the processor configured to control the wearable device, the processor including instructions to: automatically direct the transducer array to sweep an ultrasound beam across tissue when the wearable device is affixed to a surface; capture images as the ultrasound beam is sweeping; locate an image of interest from the captured images, a position of the ultrasound beam where the image of interest was collected; locate the transducer array element that collected the image of interest, where the image of interest is based on a type of test, where the transducer array element centers an area of interest. The processor includes instructions to move the ultrasound beam to a position where the image of interest was collected, where the position is associated with a beam steering angle, save the beam steering angle for grayscale imaging of a patient; and enable collecting Doppler images in proximity to the position using a Doppler angle that is different from but related to the beam steering angle. Other embodiments of this aspect include corresponding computer systems, apparatus, and computer programs recorded on one or more computer storage devices, each configured to perform the actions of the methods.

Implementations may include one or more of the following features. The system as may include: an adhesive affixed to one side of the wearable device, the adhesive configured to removably attach the one side to a surface. The transducer array elements may be individually controlled. The wearable device may include a tourniquet, wireless communications electronics, and one or more sensors. Implementations of the described techniques may include hardware, a method or process, or computer software on a computer-accessible medium.

Additional features and embodiments are set forth in part in the description which follows, or may be learned by practice of the present disclosure. It is to be understood that both the foregoing general description and the following detailed description are exemplary and explanatory and are not restrictive of the claimed subject matter.

### BRIEF DESCRIPTION OF THE DRAWINGS

The accompanying drawings, which are incorporated in and constitute a part of this specification, illustrate exemplary embodiments and together with the description, serve to explain the principles of the disclosure. In the figures:

FIG. 1A is a pictorial representation of a medical condition to which the system of the present disclosure can be applied;

FIG. 1B is a photographic representation of an exemplary wired device in accordance with embodiments of the present disclosure;

FIG. 1C is a pictorial representation of an exemplary tourniquet device in accordance with embodiments of the present disclosure;

FIG. 1D is a pictorial representation of an exemplary manufacturing process of an exemplary transducer array in accordance with embodiments of the present disclosure;

FIG. 1E is a pictorial representation of an exemplary transducer array in accordance with embodiments of the present disclosure;

FIGS. 1F and 1G are schematic block diagrams of exemplary architectures of the system in accordance with embodiments of the present disclosure;

FIG. 2A is a pictorial representation of an exemplary process overview in accordance with embodiments of the present disclosure;

FIG. 2B is a pictorial representation of an exemplary process in accordance with embodiments of the present disclosure;

FIGS. 2C and 2D are pictorial representations of exemplary processes including multiple transducers in accordance with embodiments of the present disclosure;

FIGS. 3A and 3B are flowcharts of an exemplary process in accordance with embodiments of the present disclosure;

FIGS. 4A and 4B are pictorial representations of beam steering and selective transducer element activation in accordance with embodiments of the present disclosure;

FIG. 4C is a pictorial representation of an exemplary transducer in accordance with embodiments of the present disclosure; and

FIG. 5 is a pictorial representation of an exemplary wireless patch in accordance with embodiments of the present disclosure.

### DETAILED DESCRIPTION

In order for the present disclosure to be more readily understood, certain terms are set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

Reference will now be made in detail to example implementations. These embodiments are described in sufficient detail to enable those skilled in the art to practice the system and method in accordance with embodiments of the present disclosure and it is to be understood that other embodiments may be utilized and that changes may be made without departing from the scope of the present disclosure. The following description is, therefore, merely exemplary.

An exemplary system in accordance with embodiments of the present disclosure includes a wearable ultrasound device and an ultrasound monitoring control system. The wearable ultrasound device and the control system can communicate wirelessly or through a wired connection. The wearable ultrasound device includes a transducer array, other sensors, and a communications device. The ultrasound monitoring control system includes a processor, a user interface, and applications configured to execute on the processor.

With respect to the transducer array, the transducer array elements generally include, at least, a lens, one or more matching layers, one or more piezo-ceramic elements, and one or more backing layers, stacked in that order, with the lens configured to contact the skin of a patient. The backing layer prevents the backward emitted sound waves from echoing and ringing back into the transducer for detection. The matching layer is made of material that enables an acoustic impedance gradient to promote acoustic energy from the transducer to penetrate the tissue and for the reflected waves to return to the transducer. In an aspect, the matching layer includes material that prevents unintended electrical shocks, and its thickness is ¼ wavelength of the ultrasound pulse.

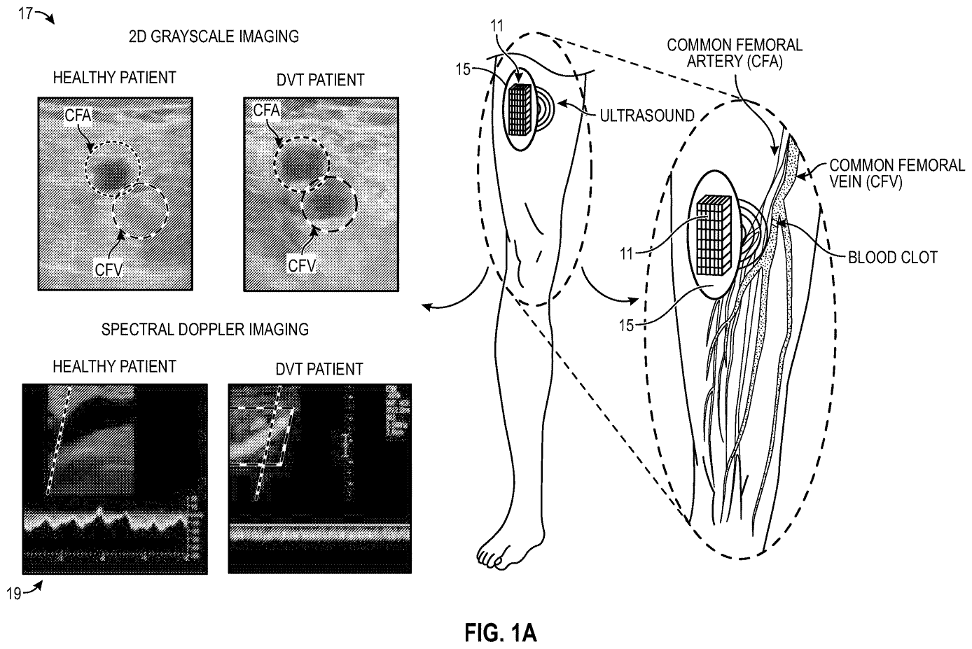

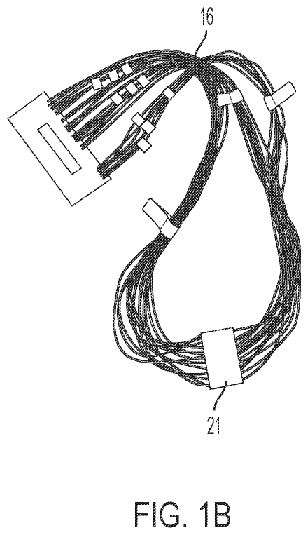

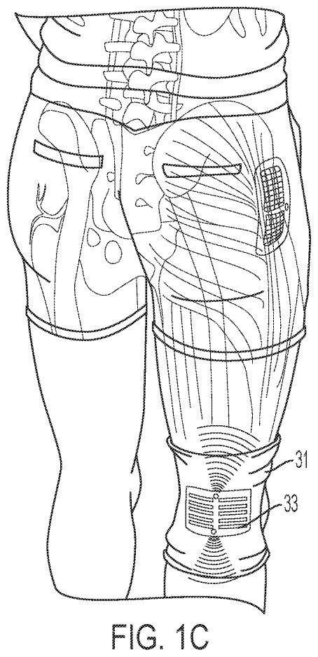

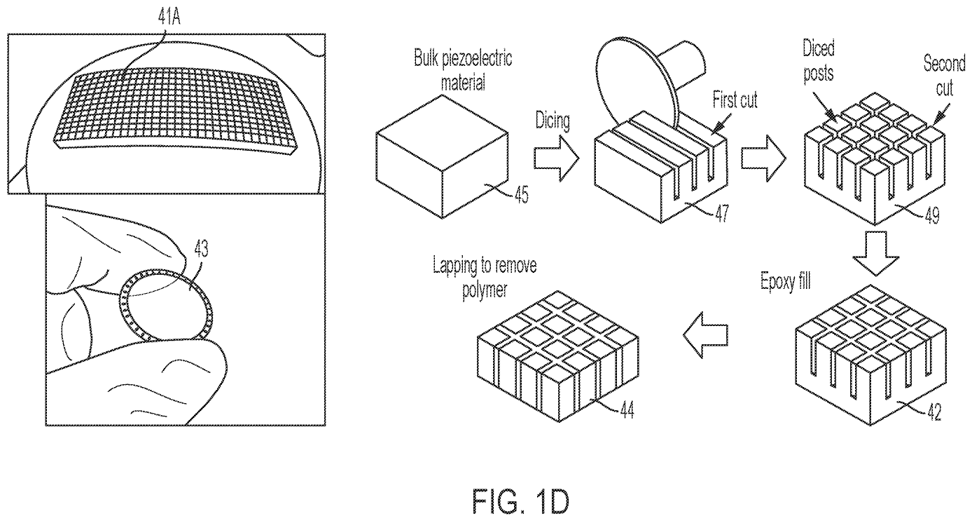

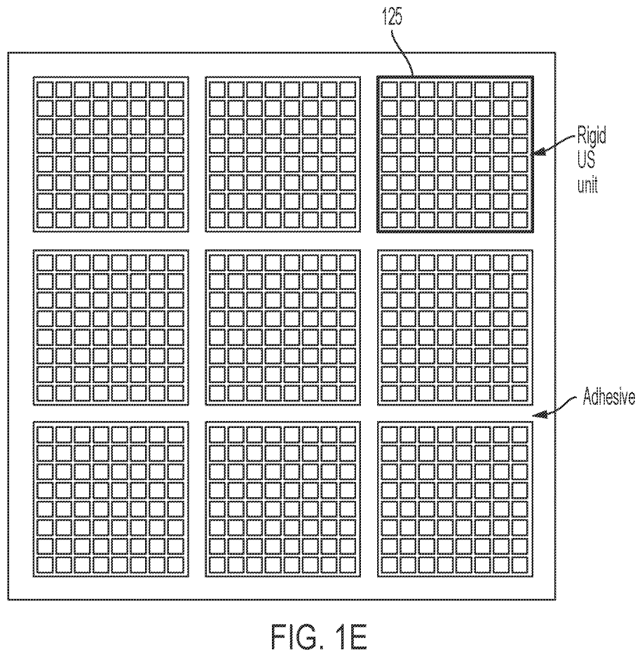

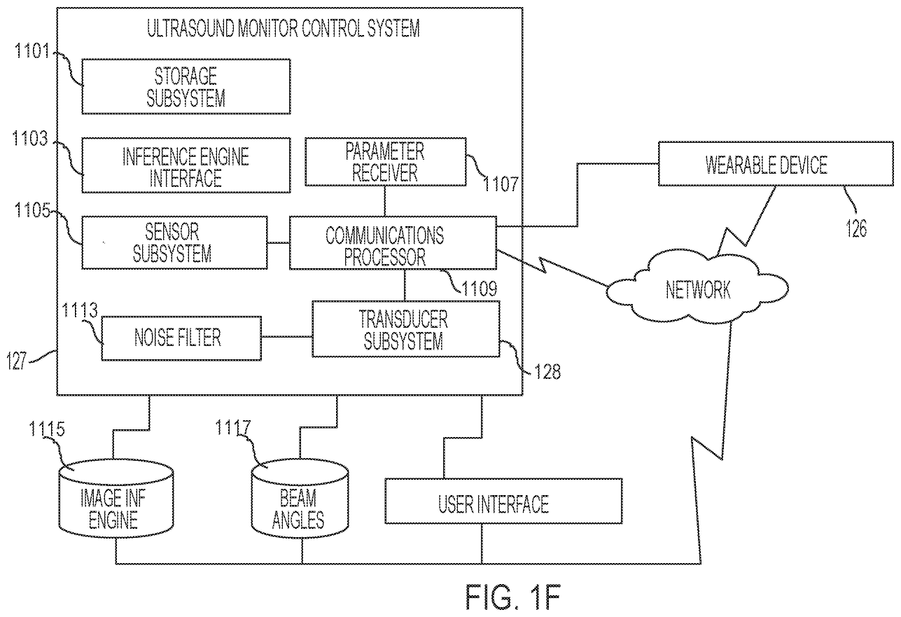

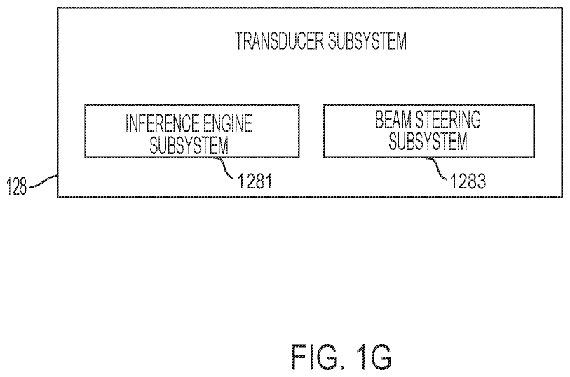

Referring now to FIGS. 1A-1C, shown are a wearable device 15 (FIG. 1A), transducer array 11 (FIG. 1A), a wearable device 21 (FIG. 1B) having wired 16 (FIG. 1B) electrical connections, and a tourniquet 31 (FIG. 1C). The shown exemplary ultrasound devices are embodiments in accordance with the present disclosure. These embodiments illustrate devices that make contact with human skin, or with a gel that is spread onto the skin. When attached directly to the skin, the wearable device 15 is constructed of material that allows adherence for an extended period of time. When the gel is the primary skin contact, the gel is formulated to allow adherence for an extended period of time. In an aspect, the wearable device 15 is formed in multiple elastomer layers with solid hydrogel between the elastomer layers, resulting in a flexible and stretchy device. In an aspect, the wearable device 15 is fabricated to wearably conform to specific anatomical regions. The elastomers prevent dehydration of the hydrogel, which is hydrated to function as a coupling mechanism. Other formulations of the wearable device, including formulations without hydrogel and elastomers, are contemplated by the present disclosure. Materials such as biodegradable plastics can be used. In an aspect, the device performs grayscale 17 (FIG. 1A) (B-mode) and blood flow 19 (FIG. 1A) (spectral Doppler) imaging synchronously.

Continuing to refer to FIGS. 1A-1C, exemplary devices in accordance with the present disclosure are configured to monitor patients on a timespan of hours to weeks, wired or wirelessly, automatically alerting clinicians of sudden changes in patient health status. In an aspect, an exemplary wearable device includes a grid or array 33 (FIG. 1C) of transducers possibly located behind the knee at the popliteal fossa, and possibly other sensors. For example, the tourniquet 31 (FIG. 1C) can include sensors that can measure compression.

Referring now to FIG. 1D, in an aspect, the wearable device, including a transducer array, is manufactured to ensure patient safety, data accuracy, and the ability to monitor variable rate blood flows, for example, slow blood flows that could be indicative of DVT risk. Specifically, the materials of the wearable device are biocompatible, and thermal and mechanical indices are calculated to prevent burning the patient and prevent cavitation, respectively. The duty cycle, gain, and pulse repetition frequency are varied to improve blood flow monitoring and reduce the risk of overheating. Further, the wearable device transducer elements are in fixed positions relative to each other, making it possible to determine element positions efficiently. Still further, the wearable device is automatically calibrated because of its geometry. The transducer array is compatible for use with both flexible and inflexible devices. The wearable device includes a backing layer that ranges from inflexible to flexible, depending upon the application.

Continuing to refer to FIG. 1D, shown are diced piezoceramic material 41A, a diamond cutting blade 43 for patterning the piezoelectric material, and a schematic diagram of the process of fabricating the transducer array in accordance with the present disclosure. A description of an exemplary process follows. After selecting a piezoelectric crystal material 45, a first step toward fabricating a 32-element 5-10 MHz ultrasound transducer array is patterning the pre-poled piezoelectric material into the pre-selected number of elements for the transducer array. The process includes cutting 47 kerfs into the piezoelectric material using, for example, a mechanical dicing saw, to section the piezoelectric material into patterned arrays 49. Piezoelectric materials can be patterned using 3D printing techniques as well. After sectioning the crystal into a transducer array, the process includes filling 42 the kerfs with epoxy to reduce cross-talk between transducers in the transducer array. The process includes lapping 44 the piezo-composite to a thickness corresponding with the pre-selected operational frequency. Electrodes are be deposited on both sides of the transducer array through electroplating or metal evaporation. Flexible wires are soldered to the transducer array and connected to an impedance-matching network in order to optimize power delivery to the transducer array. In some configurations, electromagnetic interference signals are filtered using a bandpass filter. In some configurations, electronics of embodiments of the device in accordance with the present disclosure are shielded, for example, but not limited to, with copper coating. A backing layer, for example, but not limited to, air-backed, is applied to the transducer array to ensure that the ultrasound emits forward from the transducer array surface. A matching layer, for example, but not limited to, tungsten or silver powder-loaded epoxy is applied to the front of the transducer array. Piezoelectric materials typically have an acoustic mismatch with soft tissue. Thus, the matching layer helps ensure the effective delivery of ultrasound from the device into the tissue to be imaged.

Continuing to refer to FIG. 1D, in an aspect, the operational frequency of the transducer array is determined using a combination of software packages, such as PiezoCAD, PZFlex, COMSOL, Wave3000 and K-wave. Together these software packages can simulate the acoustic pressure, the axial and lateral resolution, as well as the imaging depth, for the proposed transducer array at any given frequency. In an aspect, the mechanical properties of the transducer array are adjusted to ensure the device is able to visualize the femoral vein (mean diameter, 6.4-6.8 mm) at a minimum depth of 14.8 mm, with a maximum acoustic pressure of 720 mW/cm². The mechanical properties include, but are not limited to including, the thickness as formed by the lapping machine, and element spacing, width, and pitch.

Referring now to FIG. 1E, the device in accordance with embodiments of the present disclosure can include a schematic wearable device including a 3×3 grid of individual rigid transducers 125 placed on an adhesive. The wearable device can be flexible, semi-flexible, or inflexible, and the adhesive can be flexible, semi-flexible, or inflexible. The transducer 125 is diced into sixty-four elements (8×8). In another exemplary configuration (not shown), the transducer is diced into thirty-two elements (32×1). In an aspect, the number and configuration of transducer array elements is optimized using computer simulations of pressure field and acoustic impedance, for example.

Referring now to FIG. 1F, shown is a schematic block diagram of an exemplary ultrasound monitor control system in accordance with the present disclosure. The ultrasound monitor control system 127 includes a subsystem 1107 for receiving parameters, for example, for tuning the system. Such parameters can include noise filter values to enable the control system to distinguish between noise and slow blood flow. Another tunable parameter is the Pulse Repetition Frequency (PRF), the frequency that ultrasound pulses are emitted. In an aspect, the PRF is set to a range of approximately 1000 to 10000 Hz, which is, in general, much lower than the frequency of that the transducer array elements pulse. Another tunable parameter is the signal-to-noise ratio threshold which is adjusted so that the operational signal-to-noise ratio is usable during compression, thus ensuring the accuracy of the device under an applied static load. Other parameters that can be adjusted are gain, power, and duty cycle which settings can manage the thermal load of the wearable device 126 on the tissue. In an aspect, heat dissipation and data acquisition are optimized by, for example, varying the duty cycle, Doppler frequency, and duration of the cooldown period. In some configurations, lowering the duty cycle decreases heat dissipation. For example, in some configurations, decreasing the duty cycle to 10%, the minimum percentage to visualize spectral waveforms, decreases the temperature after 30 minutes by approximately 12 °F.

Continuing to refer to FIG. 1F, the ultrasound monitor control system 127 includes an inference engine interface 1103 (discussed with respect to FIG. 1G) and a sensor subsystem 1105 collecting and processing data from sensors located on the wearable device 126, and controlling the sensors. Distinguishing between noise and slow blood flow can be assisted by or enabled by data gathered by the sensors and processed by the sensor subsystem 1105. Other sensors can include a heat sensor measuring heat dissipated from the transducer array to the patient during use. In an aspect, the heat measurement is used to determine the maximum duration of use. For example, the wearable device 126 may be limited to an operational temperature of, for example, but not limited to, 111 °F in order to prevent the possibility of a skin burn.

Continuing to still further refer to FIG. 1F, the ultrasound monitor control system 127 includes a noise filter 1113 that receives data from the transducer array(s) and distinguishes between slow blood flow and noise in the data by use of, for example, but not limited to, frequency analysis and filtering, depending upon the data.

Continuing to refer to FIG. 1F, the ultrasound monitor control system 127 includes a communications processor 1109 that receives data from the transducer array(s) and sensor(s) on the wearable devices 125, and sends control commands to the transducer array(s) and sensor(s).

Continuing to refer to FIG. 1F, the ultrasound monitor control system 127 includes a storage subsystem 1101. In an aspect, in operation, monitored data, for example, eight bytes captured at a frequency of 30 Hz, can be stored for at least two years by, for example, commercially-available data storage options. In an aspect, the storage subsystem 1101 performs pre-selected computations, time-stamps the data, organizes the data, and saves the data in, for example, commercially-available storage, including cloud storage. For example, peak systolic velocity can be captured for a pulse, a waveform can be created, the waveform resembling a cardiac cycle, the waveform can be time-stamped, organized into, for example, a cardiac cycle category, and saved.

Referring to FIG. 1G, the transducer subsystem 128 includes a beam steering subsystem 1283 and an inference engine subsystem 1281. The inference engine subsystem 1281 is used to determine which of the transducer elements is producing an image(s) of interest so that pre-selected elements will need to be used for a particular ultrasound session. The inference engine subsystem 1281 accesses a trained neural network to locate the image(s) of interest. The neural network can include, for example, but not limited to, a supervised deep neural network based off on an object classification network (for example, but not limited to, YOLOv8) or U-Net structure is used. Grayscale images 1115 are fed to the neural network through the inference engine subsystem. For example, in the case of a femoral artery test, femoral artery and vein segmentation image(s) are output. Given a collection of segmentation outputs, the image of interest is the image that places the vessels closest to the center of the image. The transducer array element(s) associated with the image of interest is selected to fire for future data acquisitions.

Continuing to refer to FIG. 1G, the beam steering subsystem 1283 is used to steer the ultrasound beam of the selected transducer array element(s) such that anatomic and blood flow imaging are optimized. To provide a grayscale image for anatomic visualization, the ultrasound beam is positioned perpendicular to the blood vessel, thus showing a circular cross section in the ultrasound image. If the femoral vein is at an angle relative to the transducer on the skin (due to anatomic variation), the traditional forward-facing ultrasound beam may not be optimal. The beam steering subsystem 1283 selects a circular cross section of the femoral vein from the segmented vessels, and selects the beam steering angle 1117 used to create this image for future grayscale imaging of the patient. While grayscale images are acquired at a perpendicular angle, blood flow measurements via Doppler ultrasound are acquired when the ultrasound beam is parallel to the blood flow, due to the maximized Doppler shift. The beam steering subsystem 1283 selects the beam steering angle 1117 (FIG. 1F) used for color Doppler or spectral Doppler recording using a 45° angle offset from the angle chosen for grayscale imaging.

In an aspect, the beam steering subsystem 1283 interfaces with a beam controller (not shown), which includes, in an aspect, software beamforming, frame imaging using plane wave transmit beams, and RF signal data transfer to the ultrasound monitor control system. Beam steering is used to both optimize a beam angle and to gather images at the same location repetitively. In an aspect, the beam controller performs beam steering by switching between feed antenna elements. In an aspect, the beam controller performs beam steering based on activating a subset of antenna elements to steer the pattern in a pre-selected direction. In an aspect, the beam controller locates an optimum beam angle by providing the images to a first trained neural network as the beam is moved. After the optimum beam angle is determined, the images are provided to a second trained neural network. The second neural network provides an interpretation of the image itself, for example, if the image represents a known (to the neural network) pathology.

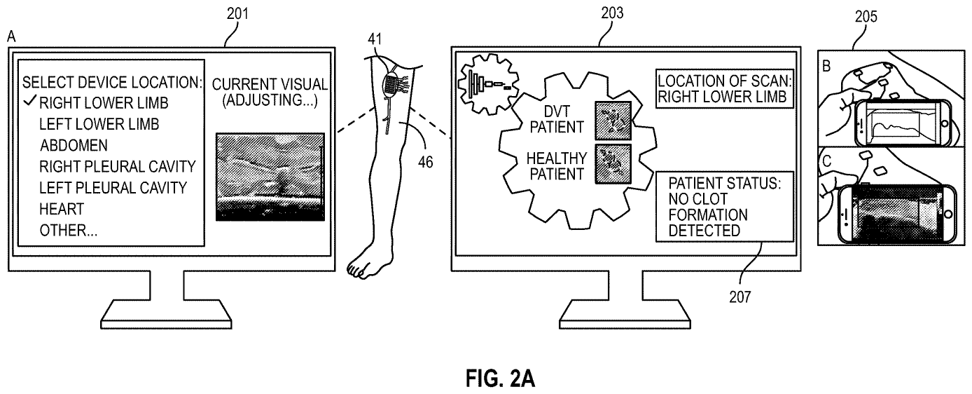

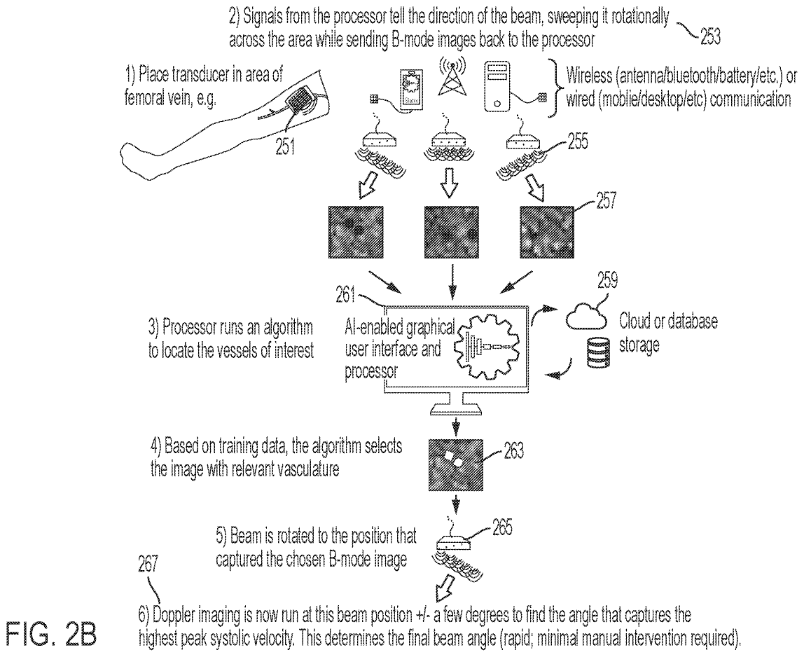

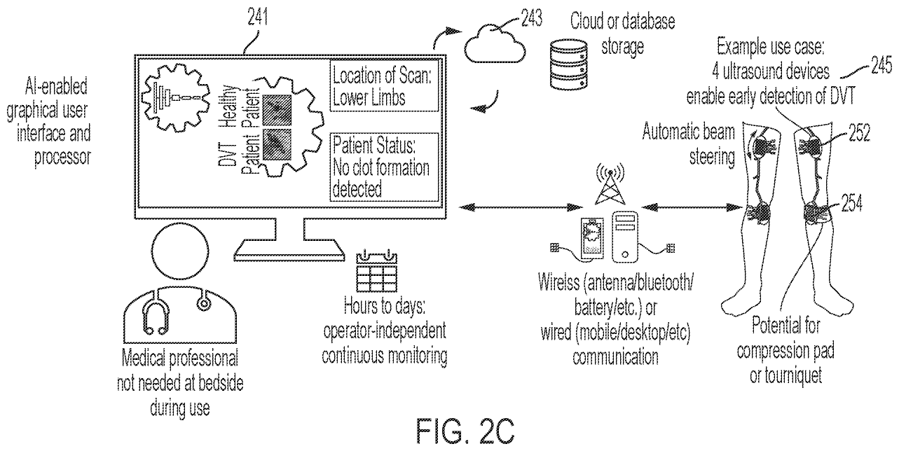

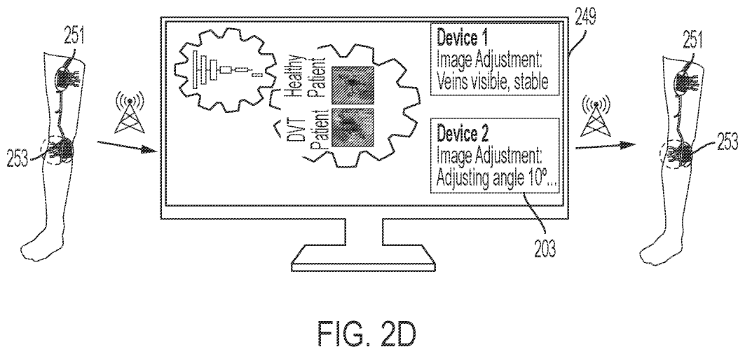

Referring now to FIG. 2A, an overview of the process that could be used to employ the wearable device in accordance with embodiments of the present disclosure is shown graphically. In FIG. 2A, shown is an exemplary sequence of events including selecting 201 a body part to which the wearable device of the present disclosure is to be affixed. Other options can be selected, as appropriate for the pre-selected diagnostic assistance and the patient's needs, among other variables. Next, the process includes receiving 203 ultrasound data from the transducer 41 affixed to the lower limb 46, viewing 205 the image created from the ultrasound data, and receiving 207 a status of the body part.

Referring now to FIG. 2B, the process that a clinician might follow to use the device of the present disclosure includes placing 251 the transducer array (wired or wireless) in the area of the body part to be scanned. When the transducer array is positioned on the patient, the ultrasound beam is swept 253 rotationally, or a change in angulation/tilt and/or change in array (e.g., linear vs. phased) are possible, starting with its original direction, across the area of the body part while sending grayscale images to a processor. The processor can include, but is not limited to including, a wired or wireless device. The processor captures the images 257 from various directions 255 and provides the images 257 to an inference engine 261 that locates images of interest based on what the user is interested in testing. For example, if the body part is a femur and the vessel of interest is a femoral vein, the images of interest would be blood vessels. To determine which of the images would be relevant to the diagnosis, the collected images are possibly stored 259 and in any case provided to an inference engine 261 that has been trained with vessel data. The inference engine selects 263 the image or images that include the relevant vasculature. The system has tracked the position where the image or images of interest were collected. With that information, the system moves 265 the ultrasound beam to the position in which the chosen image or images was captured. The system enables Doppler imaging 267 in the vicinity of the chosen position (for example, +/−a few degrees on each side of the position) to find the beam angle that captures the highest peak systolic velocity. The beam angle is at the position of the peak systolic velocity.

For example, through continuous monitoring of the data from the transducer, images collected following beam steering are provided to a predictive model, such as a deep learning model, that indicates the possibility of a particular diagnosis, such as a DVT. Such results can be used, for example, to instruct the beam steering subsystem to enable array elements in the transducer to collect data from various anatomical structures, and/or to order further diagnostic procedures as dictated by the predictive model. In an aspect, artificial intelligence techniques are used to gain an anatomical understanding of the region and also to inform beam steering. In an aspect, an object recognition network is trained on many anatomical images, including but not limited to the upper thigh region for locating the femoral artery/vein. Examples of artificial intelligence techniques that perform this function and others are Convolutional Neural Networks (CNN), Region-based Convolutional Neural networks (R-CNNs) such as Faster-RCNN, Recurrent Neural Network (RNN) such as Long Short-Term Memory (LSTM) and Gated Recurrent Unit (GRU), gradient boosting, dimensionality reduction such as principal component analysis (PCA), t-distributed Stochastic Neighbor Embedding (t-SNE) and truncated singular value decomposition (SVD), random forest, and the YOLO model family. Objects within training set images are labeled as anatomical objects (for example, bone, vein, etc.). When new images are presented to the trained network, the trained network estimates which of the objects it has seen is a likely match for the objects in the image, and provides a probability that the estimate is correct. The images can be provided collectively to a network that has been trained, or that can learn, one or more possible diagnoses and treatments that are indicated by the set of images.

Referring now to FIGS. 2C and 2D, four wired transducers are shown feeding information to a processing application executing on a specialized device formulated specifically for interpreting ultrasound data, or executing on a general purpose computing device, such as a handheld device, a laptop, a tablet, a desktop computer, and/or a mobile phone, executing specialized instructions. The communications between the transducer and the processing device can be wired (not shown) or wireless. In FIG. 2C, the data from the transducers is shown on a mobile phone screen after being received, processed, and displayed by a mobile phone application, or after being received by the sensors on a mobile phone, processed by an application executing elsewhere, for example in a remote (to the mobile phone) application, and displayed on the mobile phone that received the data or elsewhere. In an aspect, the transducer data are received by one or more computers, processed by other computers, and displayed by yet other, or multiple other displays associated with yet other computers. For example, an AI-enabled graphical user interface and processor 241 supplies data to, and receives data from, a storage device 243, such as, for example, but not limited to, cloud storage. In an exemplary use case, four ultrasound devices 245 enable early detection of DVT. Sensors 252 on the thigh or compression pad or tourniquet 254 at the knee are possible to enable DVT detection. Sensors 252/254 can supply, either wirelessly or wired, data to the graphical user interface and processor 241 by operator independent continuous monitoring. The angle of the image from a specific device can be adjusted by an application 23. In the example, a medical professional may or may not be at the bedside of the patient, but can monitor the patient's condition remotely.

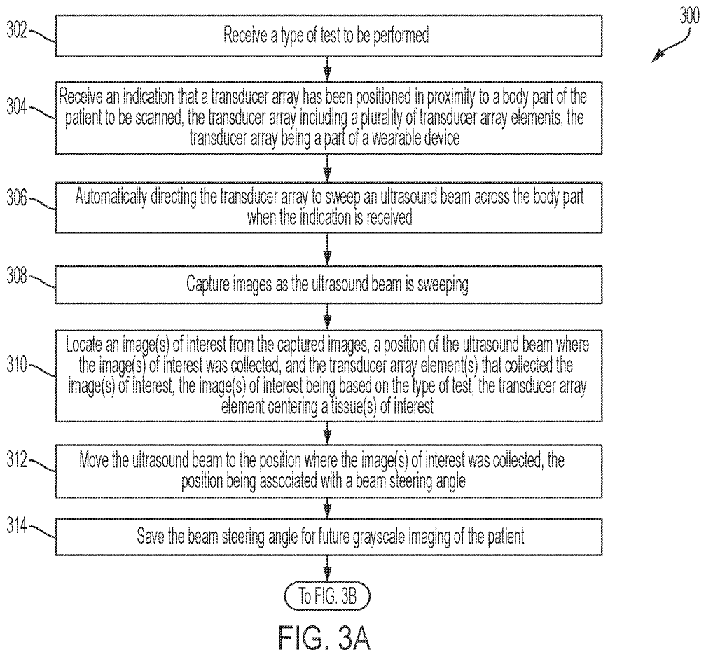

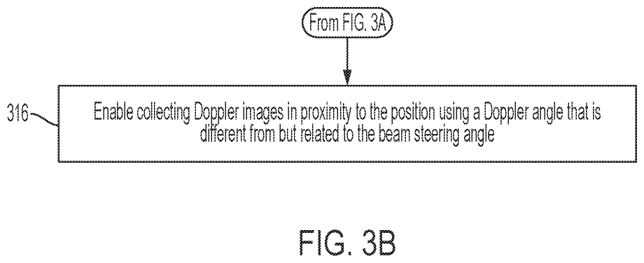

Referring now to FIGS. 3A and 3B, to enable the clinician's process, an exemplary method 300 of the present disclosure includes, but is not limited to including, receiving 302 a type of test to be performed, and receiving 304 an indication that a transducer array has been positioned on the patient in the area of the body part to be scanned. When the indication has been received that the transducer array is positioned on the patient, the method includes steering the ultrasound beam such that anatomic and blood flow imaging are optimized. In an aspect, steering the beam includes automatically directing 306 the transducer array to sweep the ultrasound beam, starting with its original direction, across the area of the body part and capturing 308 images from the various directions as the ultrasound beam is sweeping. The method includes locating 310 the image(s) of interest from the captured images, a position of the ultrasound beam where the image(s) of interest was collected, and the transducer array element(s) that collected the image(s) of interest, the image(s) of interest being based on the type of test, the transducer array element centering a tissue(s) of interest. The method includes moving 312 the ultrasound beam to the position where the image(s) of interest was collected, the position being associated with the beam steering angle. For example, if vasculature data are being scanned, the best data include the highest peak systolic velocity, for example, positioning the beam perpendicular to the blood vessel for grayscale imaging, thus showing a circular cross section in the ultrasound image. If the femoral vein is at an angle relative to the transducer on the skin (due to anatomic variation), the traditional forward-facing ultrasound beam may not be optimal. In an aspect, the method includes selecting a circular cross section of the femoral vein from the segmented vessels, and saving 314 the beam steering angle used to create this image for future grayscale imaging of the patient. The method further includes enabling collecting 316 Doppler images in proximity to the position using a Doppler angle that is different from but related to the beam steering angle, for example, +/−a few degrees on each side of the position, to find a beam angle that captures the best data. While grayscale images are typically acquired at a perpendicular angle, blood flow measurements via Doppler ultrasound are optimized when the ultrasound beam is parallel to the blood flow, due to the maximized Doppler shift. The method includes selecting the beam steering angle used for color Doppler or spectral Doppler recording using a 45° angle offset from the angle chosen for grayscale imaging.

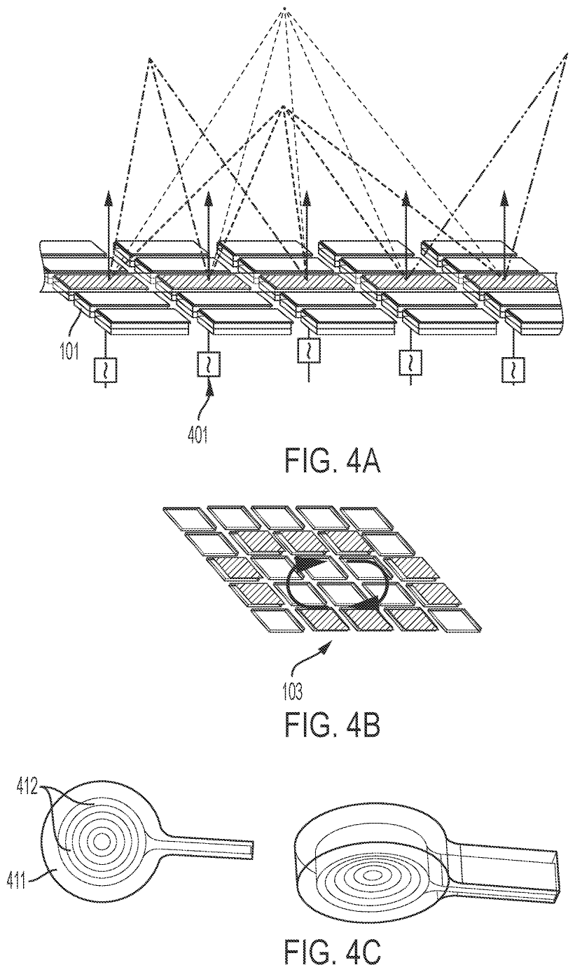

Referring now to FIGS. 4A and 4B, an exemplary configuration of the operator-independent wireless wearable ultrasonic device transducer is shown. The wearable device can be positioned by anyone of any skill and the associated software/firmware/hardware accommodates, through the use of artificial intelligence techniques, for poor positioning, and enables continuous monitoring. In an aspect, the transducer operates periodically, for example, one minute out of ten minutes, or one minute thirty minutes, etc. in order to avoid overheating. Therefore, the transducer is turned on/off intermittently in addition to the elements sending intermittent signals while imaging. Individual electronically-controlled transducer elements 101 allow a variety of beam adjustments.

Referring now to FIG. 4A, curved and linear wavefronts may be obtained by varying the timing/excitation signal 401 of elements 101, and the independent nature of elements 101 allows for grouping of any number of elements 101 to form multiple probes on-the-go. For example, the same set of elements 101 can scan one target or can be subdivided into multiple probes that can scan several targets simultaneously.

Referring now to FIG. 4B, multi-plane, quasi-three dimensional, imaging of two dimensional array 103 of elements 101 allows for simultaneous multiple views of the same target, for example, but not limited to, short and long axis views of a vessel, with potential for tomographic reconstruction revealing multi-dimensional characteristics. Quasi-three dimensional imaging refers to the capability of producing 3D images without using a 3D probe configuration. Shown in FIG. 4B is an illustration of enabling and disabling elements, and possibly changing their individual angles to produce imaging.

Referring now to FIG. 4C, shown is an exemplary ultrasonic annular array transducer 411, the shape and size of which can be modified without deviating from the scope of the present disclosure. The annular array transducer is one option, in addition to, for example, but not limited to, a linear array and matrix transducer. In an aspect, the annular array transducer is used in large thickness and high attenuation applications. These could provide dynamic focusing capabilities and symmetrical beams. The exemplary annular array transducer includes transducer elements 412, shown as concentric circles. Any shape and geometry of the transducer elements are within the scope of the present disclosure.

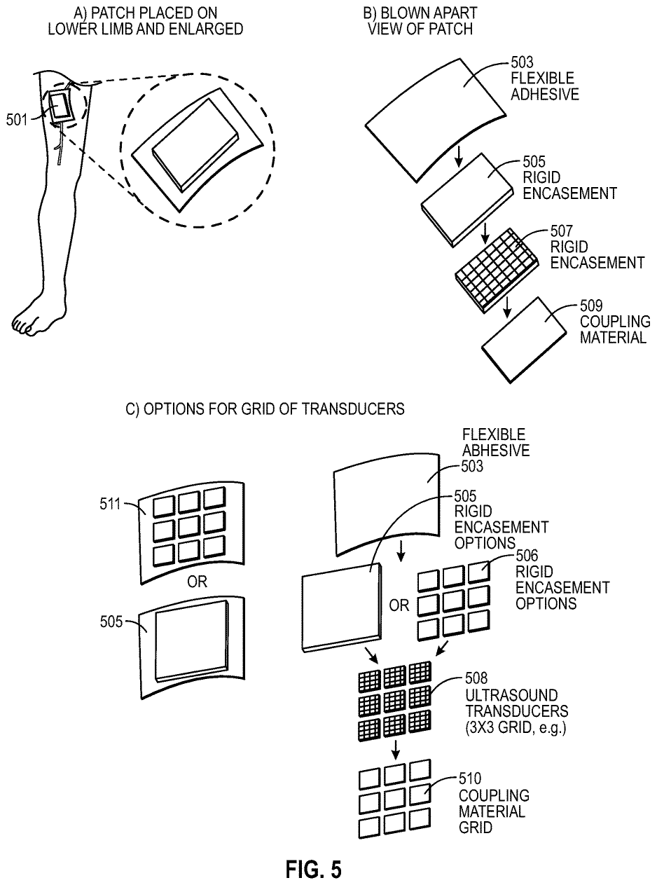

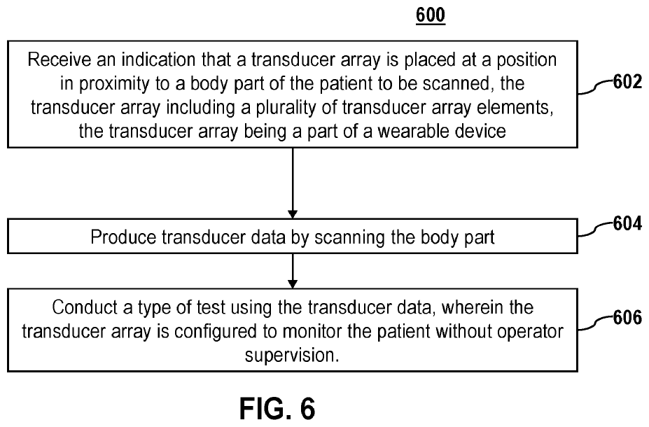

Referring now to FIG. 5, shown is an exemplary wireless patch in accordance with embodiments of the present disclosure. The wireless patch 501 includes a rigid transducer or array of transducers while the patch is flexible. Such a structure enables maintaining the relative positions of the transducer(s), which is related to beam steering. The wireless patch 501 is shown placed over the femoral vein on the lower limb. In an aspect, the patch 501 is manufactured to resemble an adhesive bandage or glucose patch, with a flexible adhesive covering an encasement and electronics.

Continuing to refer to FIG. 5, in an aspect, the wireless patch 501 includes a flexible adhesive layer 503, for example, but not limited to, a plastic or a cloth. Beneath the flexible adhesive layer 503 is a rigid encasement 505. In an aspect, the rigid encasement 505 is 3-D printed in plastic/resin/stainless steel/etc., or fabricated from, for example, but not limited to, stainless steel/titanium/acrylic or any material that does not cause reverberation of ultrasound waves. In an aspect, the rigid encasement 505 covers one or more transducers 507. The transducer 507 is then coupled to the patient with a coupling material 509 such as a hydrogel. In the case of an array of transducers 511, the individual transducer may have its own rigid encasement 506, or there could be one large encasement 505 surrounding the array, for example, a 3×3 grid of 16-element transducers 508. Coupling material 510 ensures the sound waves pass into the patient.

While embodiments in accordance with the present disclosure have been described herein, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

Other embodiments will be apparent to those skilled in the art from consideration of the specification and practice of the embodiment disclosed herein. It is intended that the specification and examples be considered as exemplary, with a true scope and spirit of the present disclosure being indicated by the following claims.

1. A method for monitoring a medical condition of a patient, the method comprising:
receiving an indication that a transducer array is placed at a position in proximity to a body part of the patient to be scanned, the transducer array including a plurality of transducer array elements, the transducer array being a part of a wearable device;
producing transducer data by scanning the body part; and
conducting a type of test using the transducer data,
wherein the transducer array is configured to monitor the patient without operator supervision.

2. The method as in claim 1 wherein producing transducer data by scanning the body part comprises:
automatically directing the transducer array to sweep an ultrasound beam across the body part when the indication is received;
capturing images as the ultrasound beam is sweeping;
locating an image of interest from the captured images, a position of the ultrasound beam where the image of interest was collected, and a collecting transducer array element from the plurality of transducer array elements that collected the image of interest, the image of interest being based on the type of test, the collecting transducer array element centering a tissue of interest; and
moving the ultrasound beam to the position where the image of interest was collected,
wherein the position is associated with a beam steering angle.

3. The method as in claim 2 further comprising:
saving the beam steering angle for future grayscale imaging of the patient.

4. The method as in claim 2 further comprising:
enabling collecting Doppler images in proximity to the position using a Doppler angle that is different from but related to the beam steering angle.

5. The method as in claim 1 wherein the proximity position is a popliteal fossa.

6. The method as in claim 1 wherein the wearable device comprises a backing layer.

7. The method as in claim 1 wherein the wearable device comprises automatic calibration.

8. The method as in claim 1 wherein the wearable device comprises wireless communications electronics.

9. The method as in claim 1 wherein the wearable device comprises a sensor.

10. The method as in claim 1 wherein the wearable device comprises a tourniquet.

11. The method as in claim 2 wherein locating the image(s) of interest comprises:
providing the image to an inference engine; and
receiving information from the inference engine, the information enabling identification of the image(s) of interest.

12. The method as in claim 2 further comprising:
providing the image of interest to a predictive model; and
receiving a diagnosis and a probability that the diagnosis is correct from the predictive model.

13. The method as in claim 12 further comprising:
using the diagnosis to activate/deactivate selective of the plurality of transducer array elements.

14. A wearable ultrasound device for monitoring of tissue comprising:
a transducer array including a plurality of transducer array elements and a plurality of subdice elements;
a thermal sensor configured to monitor a temperature of the wearable ultrasound device having a pre-selected thickness and spacing between elements; and
communications electronics receiving control information to control the plurality of transducer array elements,
wherein the control information enables monitoring of the plurality of transducer array elements, and
wherein the communications electronics transmits data from the thermal sensor and the plurality of transducer array elements to a processor.

15. The wearable ultrasound device as in claim 14 comprising:
an acoustic lens.

16. The wearable ultrasound device as in claim 14 wherein the control information comprises beam steering information including instructions to:
automatically direct the transducer array to sweep an ultrasound beam across the tissue when the wearable ultrasound device is affixed to a surface; and
save a beam steering angle for grayscale imaging of a patient.

17. The wearable ultrasound device as in claim 16 wherein the beam steering information comprises instructions to:
capture images as the ultrasound beam is sweeping;
locate an image of interest from the captured images, a position of the ultrasound beam where the image of interest was collected;
identify transducer array elements from the plurality of transducer array elements that collected the image of interest,
wherein the image of interest is based on a type of test,
wherein the identified transducer array elements center an area of interest; and
move the ultrasound beam to the position where the image of interest was collected,
wherein the position is associated with a beam steering angle.

18. The wearable ultrasound device as in claim 17 wherein the control information enables collecting Doppler images in proximity to the position using a Doppler angle that is different from but related to the beam steering angle.

19. The wearable ultrasound device as in claim 14 wherein the communications electronics comprise wireless electronics.

20. The wearable ultrasound device as in claim 14 wherein the control information comprises:
duty cycle information for the transducer array elements, the duty cycle information based on data from the thermal sensor.

21. A system for ultrasonic monitoring comprising:
a wearable device configured to collect ultrasound data from a user wearing the wearable device, the wearable device including:
a transducer array including a plurality of transducer array elements;
a thermal sensor configured to monitor a temperature of the wearable device; and
communications electronics receiving control information for the transducer array elements, the control information enabling the ultrasonic monitoring, the communications electronics transmitting data from the thermal sensor and the transducer array elements to a processor, the processor configured to control the wearable device, the processor including instructions to:
automatically direct the transducer array to sweep an ultrasound beam across tissue when the wearable device is affixed to a surface;
capture images as the ultrasound beam is sweeping;
locate an image of interest from the captured images, a position of the ultrasound beam where the image of interest was collected;
locate the transducer array element that collected the image of interest,
wherein the image of interest is based on a type of test,
wherein the transducer array element centers an area of interest;
move the ultrasound beam to a position where the image of interest was collected,
wherein the position is associated with a beam steering angle;
save the beam steering angle for grayscale imaging of a patient; and
enable collecting Doppler images in proximity to the position using a Doppler angle that is different from but related to the beam steering angle.

22. The system as in claim 21 comprising:
an adhesive affixed to one side of the wearable device, the adhesive configured to removably attach the one side to a surface.

23. The system as in claim 21 wherein the transducer array elements are individually controlled.

24. The system as in claim 21 wherein the wearable device comprises a tourniquet.

25. The system as in claim 21 wherein the wearable device comprises wireless communications electronics.

26. The system as in claim 21 wherein the wearable device comprises a sensor.
